(19) United States
(12) Patent Application Publication
(10) Pub. No.: US 2007/0065393 A1
(43) Pub. Date: Mar. 22, 2007

(54) Make-up-removing composition

(75) Inventor: Arnaud Bonafos, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/516,492

(22) Filed: Sep. 7, 2006

Related U.S. Application Data
(60) Provisional application No. 60/723,006, filed on Oct. 4, 2005.

(30) Foreign Application Priority Data
Sep. 20, 2005 (FR) 05 52821

(51) Int. Cl. A61K 8/37

(52) U.S. Cl. 424/70.31

(57) ABSTRACT

An aqueous make-up-removing composition containing a derivative (product) of polyethylene glycol and a mixture of glycerides of caprylic acid and of capric acid comprising 7 ethylene oxide groups. The composition can constitute a make-up-removing water which is very comfortable to the eyes, used in particular for removing make-up gently from the skin and/or from the region of the eyes.

### REFERENCE TO PRIOR APPLICATIONS

This application claims priority to U.S. provisional application Ser. No. 60/723,006 filed Oct. 4, 2005, and to French patent application 0552821 filed Sep. 20, 2005, both incorporated herein by reference.

### FIELD OF THE INVENTION

The invention relates in a preferred embodiment to an aqueous composition comprising PEG-7 caprylic/capric glycerides, and the use of the composition for, e.g, removing make-up from the skin and/or eyes. The invention also relates to the cosmetic use of the invention surfactant for removing make-up from the eyes and/or skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

A person using mascaras, foundations or lipsticks, in particular long-lasting and waterproof products, is on the lookout for products which make possible satisfactory removal of make-up with optimum comfort on use, that is to say having both good cosmetic qualities and good tolerance. She is thus looking for a make-up-removing product which is satisfactorily effective in removing make-up while -being gentle, pleasant to use and well tolerated, that is to say not causing irritation.

The products currently provided often comprise a large amount of oil, the presence of oil making it possible to obtain good make-up-removing effectiveness. However, these products have the disadvantage of leaving an unpleasant and unsightly greasy film on the skin. Furthermore, there exist aqueous make-up-removing compositions which comprise surfactants and which are devoid of oil but the effectiveness of which with regard to long-lasting make-up is limited and often inadequate.

The need thus remains to have available an aqueous make-up-removing composition which has an improved make-up-removing power while being very comfortable for the skin and eyes and while thus having good tolerance and in particular good tolerance by the eyes, the region of the eyes being particularly sensitive.

### SUMMARY OF THE INVENTION

The inventor has now discovered, surprisingly, that a derivative (product) of polyethylene glycol and a mixture of glycerides (mono-, di- and triglycerides) of caprylic acid and of capric acid comprising 7 ethylene oxide groups (CTFA name: PEG-7 caprylic/capric glycerides) in a limited amount makes it possible to solve the above-noted problem and to obtain aqueous compositions devoid of oils which have a very good make-up removing effectiveness, in particular with regard to long-lasting foundations, in comparison with surfactants currently used in make-up removal.

### DETAILED DESCRIPTION OF THE PREFERRED EMBODIMENTS

In a preferred embodiment the present invention relates to an aqueous composition, preferably a make-up-removing cosmetic composition, comprising a derivative (product)of polyethylene glycol and a mixture of glycerides of caprylic acid and of capric acid comprising 7 ethylene oxide groups in an amount of less than or equal to 10 by weight, with respect to the total weight of the composition.

This composition is devoid of oil. Indeed, the term “aqueous composition” is understood here to mean a composition devoid of oil. The aqueous composition of the invention generally comprises at least 55% by weight of water, preferably at least 65% by weight of water and even better still at least 75% by weight of water, with respect to the total weight of the composition. The amount of water can range in particular from 55 to 99% by weight, preferably from 65 to 98% by weight and better still from 75 to 98% by weight, with respect to the total weight of the composition. The composition obtained is generally clear, that is to say transparent to translucent. Its transparency can be measured, for example, by a coefficient of transmission at 600 nm ranging from 10 to 90% or else by a turbidity ranging from 60 to 600 NTU and preferably from 70 to 300 NTU, which turbidity is measured with an Hach model 2100 P portable turbidimeter.

The composition obtained can constitute in particular a make-up-removing water of lotion type or a gel. The word “lotion” means that the composition is liquid and has a viscosity of less than 0.1 Pa·s (100 centi-poises), preferably less than 0.06 Pa·s (60 centi-poises), this viscosity being measured at 25° C. with a Rheomat RM 180 device from Rheometric Scientific, this device being equipped with a different spindle depending on the viscosities, for example with a spindle 1 or 2 for viscosity ranges of less than 7 poises, with a spindle 3 for viscosity ranges from 2 to 40 poises and with a spindle 4 for viscosity ranges from 20 poises to 100 poises.

A gel can have a viscosity ranging up to 5 Pa·s. The viscosity of the gels according to the invention preferably ranges from 0.1 to 200 poises (0.01 to 20 Pa·s) and better still from 5 to 150 poises (0.5 to 15 Pa·s), measured at 25° C. using the Rheomat RM 180.

The composition according to the invention is preferably intended and adapted for topical application and thus preferably comprises a physiologically acceptable medium. The term “physiologically acceptable medium” is understood to mean a medium compatible with the skin, mucous membranes (including the inside of the eyelids, the lips), nails, region of the eyes and/or keratinous fibres (hair and eyelashes).

Unexpectedly, the derivative (product) of polyethylene glycol and the mixture of glycerides of caprylic acid and of capric acid comprising 7 ethylene oxide groups, even when present in an amount of less than or equal to 10% and even, preferably, in an amount of less than 5% of the total weight of the composition, makes possible good removal of make-up from keratinous substances and in particular from the skin or from the region of the eyes (eyelashes, eyelids). It makes possible, for example, good removal of the mascara which is found on the eyelashes, good removal of the eyeliner which is found on the eyelids and good removal of the foundation which is found on the skin.

Consequently, a further subject-matter of the invention is the cosmetic use of a derivative (product) of polyethylene glycol and a mixture of glycerides of caprylic acid and of capric acid comprising 7 ethylene oxide groups in an amount of less than or equal to 10% by weight, with respect to the total weight of the composition, for removing make-up from the skin and from the region of the eyes.

Mention may in particular be made, as derivative (product) of polyethylene glycol and a mixture of glycerides of caprylic acid and of capric acid comprising 7 ethylene oxide groups or PEG-7 caprylic/capric glycerides, of the product sold under the name Cetiol HE 810 by Cognis.

The amount of PEG-7 caprylic/capric glycerides is not limited and can range, for example, from 0.01 to 10% by weight, preferably from 0.05 to 10% by weight, better still from 0.05 to 5% by weight and even better still from 0.5 to 5% by weight, with respect to the total weight of the composition.

In addition, the aqueous composition of the invention can comprise one or more surfactants other than PEG-7 caprylic/capric glycerides. The total amount of surfactants (PEG-7 caprylic/capric glycerides+other surfactants) preferably ranges from 0.01 to 12% by weight, more preferably from 0.05 to 10% by weight, more preferably still from 2 to 10% by weight and better still from 3 to 10% by weight, with respect to the total weight of the composition.

Use may be made, as other surfactant, of any surfactant commonly used in make-up-removing compositions. The surfactant can be chosen from nonionic surfactants, anionic surfactants, amphoteric or zwitterionic surfactants and their mixtures.

Mention may be made, as additional surfactants which can be used in the composition according to the invention, of:

(1) among nonionic surfactants:
oxyethylene/oxypropylene block polymers, such as the poloxamers and in particular poloxamer 184 and poloxamer 124 (CTFA name);
sorbitan fatty acid esters and their oxyethylenated derivative (product)s, such as the sorbitan monostearate (CTFA name: sorbitan stearate) sold by ICI under the name Span 60, the sorbitan monopalmitate (CTFA name: sorbitan palmitate) sold by ICI under the name Span 40, the oxyethylenated sorbitan stearates, palmitates and oleates (CTFA name: polysorbate) sold by ICI under the Tween names, in particular polysorbate 60 (Tween 60), polysorbate 65 (Tween 65), polysorbate 80 (Tween 80);
derivative (product)s of polyethylene glycol and of a mixture of glycerides (mono-, di- and triglycerides) of caprylic acid and of capric acid having 6 or 8 ethylene oxide groups, such as that comprising 6 ethylene oxide groups (CTFA name: PEG-6 caprylic/capric glycerides) sold under the name Softigen 767 by Sasol and that comprising 8 ethylene oxide groups (CTFA name: PEG-8 caprylic/capric glycerides) sold under the name L.A.S. by Gattefosse;
derivative (product)s of polyethylene glycol and of a mixture of glycerides (alkoxylated triglycerides) of vegetable origin, such as, for example, PEG-10 olive glycerides, PEG-45 palm kernel glycerides, PEG-20 almond glycerides,
alkylpolyglycosides and in particular alkylpoly-glucosides (APG) having an alkyl group comprising from 6 to 30 carbon atoms (alkyl-C₆-C₃₀-polyglucosides) and preferably 8 to 16 carbon atoms, such as, for example, decyl glucoside (alkyl-C₉/C₁₁-polyglucoside (1.4)), such as the product sold under the name Mydol 10 by Kao Chemicals, the product sold under the name Plantaren 2000 UP or Plantacare 2000 UP by Henkel and the product sold under the name Oramix NS 10 by Seppic; caprylyl/capryl glucoside, such as the product sold under the name Oramix CG 110 by Seppic; lauryl glucoside, such as the products sold under the names Plantaren 1200 N and Plantacare 1200 by Henkel; and coco glucoside, such as the product sold under the name Plantacare 818/UP by Henkel;

(2) among anionic surfactants:
alkyl sulphates and their salts, alkyl ether sulphates and their salts, in particular their sodium salts, such as the sodium laureth sulphate/magnesium laureth sulphate/sodium laureth-8 sulphate/magnesium laureth-8 sulphate mixture sold under the name of Texapon ASV by Henkel; the sodium lauryl ether sulphate (C₁₂₋₁₄ 70/30) (2.2 EO) sold under the names Sipon AOS 225 or Texapon N702 PATE by Henkel; the ammonium lauryl ether sulphate (C₁₂₋₁₄ 70/30) (3 EO) sold under the name Sipon LEA 370 by Henkel; the ammonium alkyl (C₁₂-C₁₄) ether (9 EO) sulphate sold under the name Rhodapex AB/20 by Rhodia Chimie;

(3) among amphoteric or zwitterionic surfactants, alkyl amphoacetates, such as the N-disodium N-cocoyl-N-carboxymethoxyethyl-N-(carboxymethyl)ethylenediamine (CTFA name: disodium cocoamphodiacetate) sold in saline aqueous solution under the name Miranol C2M Conc. NP by Rhodia Chimie; N-sodium N-cocoyl-N-hydroxyethyl-N-(carboxymethyl)ethylenediamine (CTFA name: sodium cocoamphoacetate); cocamides, such as the mixture of coconut acid ethanolamides (CTFA name: cocamide DEA).

According to a preferred embodiment of the invention, the additional surfactant or surfactants are chosen from nonionic surfactants, in particular from oxyethylene/oxypropylene block polymers, polyoxy-ethylenated sorbitan esters, derivative (product)s of polyethylene glycol and of a mixture of glycerides of caprylic acid and of capric acid having 6 or 8 ethylene oxide groups, and their mixtures.

According to a particularly preferred embodiment of the invention, the composition according to the invention comprises, as other surfactant, at least one derivative (product) of polyethylene glycol and of a mixture of glycerides of caprylic acid and of capric acid having 6 ethylene oxide groups. This combination makes it possible to obtain very good make-up-removing results with very good tolerance and good comfort on use.

In the composition of the invention, the water used can be pure demineralized water but also mineral water and/or thermal water and/or sea water, that is to say that the water of the composition can be partially or completely composed of a water chosen from mineral waters, thermal waters, sea waters and their mixtures. In general, a mineral water is suitable for consumption, which is not always the case with a thermal water. Each of these waters comprises, inter alia, trace elements and/or dissolved minerals. These waters are known to be employed for specific treatment purposes depending on the specific minerals and trace elements which they comprise, such as the moisturizing and the desensitizing of the skin or the treatment of certain dermatoses. The terms “mineral waters” or “thermal waters” will denote not only natural mineral or thermal waters but also natural mineral or thermal waters enriched in additional mineral constituents and/or in additional trace elements, and also aqueous mineral solutions and/or aqueous solutions comprising trace elements prepared from purified water (demineralized or distilled).

A natural thermal or mineral water used according to the invention can, for example, be chosen from water from Vittel, water from the Vichy basin, water from Uriage, water from La Roche Posay, water from La Bourboule, water from Enghien-les-Bains, water from Saint Gervais-les-Bains, water from Neris-les-Bains, water from Allevard-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene.

Use may also be made of sea waters, such as Dead Sea water or sea bottom water.

The composition according to the invention can optionally comprise a monohydric C₁-C₃ alcohol, in particular ethanol. According to a preferred embodiment of the invention, if the composition comprises such an alcohol, the amount of alcohol is less than 5% and preferably less than 3% when a make-up remover for the eyes is involved.

The composition of the invention can comprise one or more polyols and in particular glycols. Mention may in particular be made, among the polyols which can be used in the composition according to the invention, of propylene glycol, butylene glycol, glycerol, hexylene glycol, polyethylene glycols, such as PEG-8, dipropylene glycol and their mixtures. The amount of polyol(s) in the composition of the invention is preferably such that it does not confer a tacky nature on the final composition. This amount generally ranges from 0.05 to 20% by weight and preferably from 0.1 to 15% by weight and better still from 0.5 to 10% by weight, with respect to the total weight of the composition.

The cosmetic compositions of the invention can additionally comprise adjuvants which are standard in the cosmetics field, such as antioxidants, fragrances, peptizing agents of fragance, screening agents, colouring materials or hydrophilic or lipophilic active principles. The nature of the adjuvants and their amounts have to be such that they do not modify the properties of the composition according to the invention. The amounts of these adjuvants are those conventionally used in the cosmetics field, for example from 0.001 to 10% of the total weight of the composition.

Mention may be made, as active principles which can be used in the composition of the invention, for example, of soothing agents, such as allantoin and bisabolol; floral waters, such as lime water or cornflower water; glycyrrhetinic acid and its salts, antibacterials, such as octopirox, triclosan and triclocarban; essential oils; vitamins, such as, for example, retinol (vitamin A), ascorbic acid (vitamin C), tocopherol (vitamin E), niacinamide (vitamin PP or B3), panthenol (vitamin B5) and their derivative (product)s, such as, for example, the esters of these vitamins (palmitate, acetate, propionate), magnesium ascorbyl phosphate, glycosylated vitamin C or glucopyranosylascorbic acid (ascorbyl glucoside); coenzymes, such as coenzyme Q10 or ubiquinone and coenzyme R or biotin; protein hydrolysates; plant extracts and in particular plankton extracts; and their mixtures.

The composition of the invention can also comprise hydrophilic gelling agents, such as, for example, modified or unmodified carboxyvinyl polymers, such as the products sold under the names Carbopol (INCI name: carbomer) and Pemulen (INCI name: acrylates/ClO-30 alkyl acrylate crosspolymer) by Noveon; polyacrylamides; optionally crosslinked and/or neutralized polymers and copolymers of 2-acrylamido-2-methylpropanesulphonic acid, such as the poly(2-acrylamido-2-methylpropanesulphonic acid) sold by Hoechst under the name “Hostacerin AMPS” (INCI name: ammonium polyacryldimethyltaurate); polysaccharides, such as xanthan gum, guar gum, alginates, modified or unmodified celluloses, fucose derivative (product)s, such as those sold under the names Fucogel 1000 and Fucogel 1000 PP by Solabia. The amount of these polymers can range, for example, from 0.05 to 3% by weight, with respect to the total weight of the composition.

The compositions of the invention are particularly suitable for removing make-up from the skin and/or the region of the eyes.

Consequently, a further subject-matter of the invention is the cosmetic use of the composition as defined above for removing make-up from the skin and/or the region of the eyes.

The following examples are given by way of illustration of the invention and do not have a limiting nature. All the amounts are given as percentage by weight of starting material, with respect to the total weight of the composition.

### EXAMPLE 1

according to the invention: Make-up-removing water

| | |
|---|---|
| PEG-7 caprylic/ | 1% |
| capric glycerides | |
| Water | q.s. for 100% |

Procedure:

the PEG-7 caprylic/capric glycerides and the water are mixed at ambient temperature.

A clear make-up-removing water is obtained which makes possible very good removal of make-up from the skin, in particular from the skin of the face, and the eyes.

### EXAMPLE 2

according to the invention: Make-up-removing water

| | |
|---|---|
| PEG-7 caprylic/capric glycerides | 0.5% |
| PEG-6 caprylic/capric glycerides | 0.5% |
| Water | q.s. for 100% |

Procedure:

the PEG-7 caprylic/capric glycerides, the PEG-6 caprylic/capric glycerides and the water are mixed at ambient temperature.

A clear make-up-removing water is obtained which makes possible very good removal of make-up from the skin, in particular from the skin of the face, and the eyes.

### EXAMPLE 3

according to the invention: Make-up-removing water

| | |
|---|---|
| Preservatives | 0.7% |
| Fragrance | 0.01% |
| Polysorbate 80 | 1% |
| PEG-6 caprylic/ | 1% |
| capric glycerides | |
| Poloxamer 184 | 2% |
| Poloxamer 124 | 2% |
| PEG-7 caprylic/ | 1% |
| capric glycerides | |
| Water | q.s. for 100% |

Procedure:

the surfactants (polysorbate 80, PEG-6 caprylic/capric glycerides, poloxamer 184, poloxamer 124 and PEG-7 caprylic/capric glycerides) are mixed, then the fragrance is added to this mixture and the entire combination is mixed with the water comprising the preservatives.

A clear make-up-removing water is obtained which makes possible very good removal of make-up from the skin, in particular from the skin of the face, and the eyes.

### Comparison with Other Surfactants

### COMPARATIVE EXAMPLES 4 TO 7

these comparative examples correspond to Example 1 in which the PEG-7 caprylic/capric glycerides has been replaced by an equivalent amount of another surfactant.

| Composition | 1% of surfactant | Water |
|---|---|---|
| Example 1 of the | PEG-7 caprylic/ | q.s. for 100% |
| invention | capric glycerides | |
| Comparative Example 4 | Polysorbate 80 | q.s. for 100% |
| Comparative Example 5 | PEG-6 caprylic/ | q.s. for 100% |
| | capric glycerides | |
| Comparative Example 6 | Poloxamer 184 | q.s. for 100% |
| Comparative Example 7 | Poloxamer 124 | q.s. for 100% |

The comparative test focusing on the make-up-removing power of Example 1 of the invention and of the comparative examples was carried out according to the protocol described below.

1. Materials/Equipment
Make-up-removing composition
Household soap
1 masking card cut out of a Bristol board of 4×4 cm+1 Lumicolor permanent S felt-tipped pen
CR300 colorimeter (measurement of the L.a.b colorimetry)
Air Wear LSF 12 transfer-free foundation
8 small watch glasses+small spatulas
1 accurate balance
1 stopwatch

2. Protocol
a- Mark out 4 areas on the skin of the arms (2 per arm) using the felt-tipped pen and the masking card
b- Clean the skin with the soap, rinse and dry (the marks from the felt-tipped pen are then toned down so as to prevent any transfer of ink from the felt-tipped pen into the foundation at the moment of application)
c- Measure the bare skin by colorimetry: 3 L,a,b measurements per area
d- Evenly apply 16 mg +/−0.05 mg of foundation in the 4 delimited areas and wait 30 minutes
e- Measure the skin with foundation by colorimetry: 3 L,a,b measurements per area
f- Weigh out in the watch glasses 4×208 mg +/−1 mg of make-up-removing product to be tested
g- Remove make-up from the area with the finger by a circular movement for 10 seconds and then rinse with tap water (warm; hardness not controlled) while lightly brushing with the fingers of the available hand. Do this to the 4 areas without waiting.
h- Dab with a paper handkerchief of Kleenex type and wait 15 minutes (feeling of dry skin)
i- Measure by colorimetry the skin from which the make-up has been removed: 3 L,a,b measurements per area

3. Calculation of the percentage of removal of make-up
a- Colorimetric difference between the made-up skin and the bare skin=ΔEmax:
   ΔEmax=√(Δa₁²+Δb₁²+ΔL₁²) with
   Δa₁=a bare skin—a foundation skin
   Δb₁=b bare skin—b foundation skin
   ΔL₁=L bare skin—L foundation skin
b- For a make-up-removing product: colorimetric difference between the skin from which the make-up has been removed (remove skin) and the made-up skin=ΔE for each area:
   ΔE=√(a₂²+Δb₂²+ΔL₂² with
   Δa₂=a remove skin—a foundation skin
   Δb₂=b remove skin—b foundation skin
   ΔL₂=L remove skin—L foundation skin

The mean percentage of removal of make-up corresponds to the mean of the 4 values of (ΔE/ΔEmax)*100 for a given make-up-removing product.

The results of the percentage of removal of make-up (percentage of make-up removed) for each composition are given in the table below.

| | Percentage of removal of |
|---|---|
| Composition | make-up |
| Example 1 according to the | 71% |
| invention | |
| Comparative Example 4 | 27% |
| Comparative Example 5 | 47% |
| Comparative Example 6 | 32% |
| Comparative Example 7 | 22% |

This table shows that the composition according to the invention is much more effective in removing make-up than the compositions of the comparative examples, even in comparison with the composition of Comparative Example 5, which comprises a surfactant (PEG-6 caprylic/capric glycerides) having a structure analogous to that of PEG-7 caprylic/capric glycerides.

### Comparison with Compositions Comprising Make-up-removing Oils

The make-up-removing effectiveness of Example 1 according to the invention was evaluated in comparison with make-up-removing milks which comprise oils.

The make-up-removing milks had the following compositions:

### COMPARATIVE EXAMPLE 8

Phase A:

| Water | q.s. for 100% |
|---|---|
| Preservatives | 0.2% |
| Glycerol | 5% |
| Cleaning surfactants | 1.5% |
| (disodium coco-amphodiacetate/ | |
| sodium laureth sulphate) | |
| Xanthan gum | 0.1% |

Phase B:

| | |
|---|---|
| Liquid petrolatum | 12% |
| Isopropyl palmitate | 7.5% |
| Cetearyl alcohol | 0.4% |
| Ceteareth-30 | 0.1% |
| Carbomer | 0.6% |

Phase C:

| | |
|---|---|
| Water | 5% |
| NaOH | q.s. for pH 6.5 |

### COMPARATIVE EXAMPLE 9

Phase A:

| | |
|---|---|
| Water | q.s. for 100% |
| Preservatives | 0.2% |
| Glycerol | 3% |
| Cleaning surfactants | 1.5% |
| (disodium coco-amphodiacetate/ | |
| sodium laureth sulphate) | |

Phase B:

| | |
|---|---|
| Ethylhexyl palmitate | 15% |
| Pemulen TR-2 | 0.15% |

Phase C:

| | |
|---|---|
| Water | 5% |
| NaOH | q.s. for pH 6.5 |

The make-up-removing power for the eyes was evaluated according to the same test as that described for Comparative Examples 4-7.

| | Percentage of removal of |
|---|---|
| Composition | make-up |
| Example 1 according to the | 71% |
| invention | |
| Comparative Example 8 | 19% |
| Comparative Example 9 | 26% |

These results show that the composition according to the invention is much more effective in removing make-up than make-up-removing milks respectively comprising 19.5% and 15% of make-up-removing oils, which are the levels commonly used in make-up-removing milks.

Tests on a Panel

Furthermore, two in vivo tests were carried out with the composition of Example 3 according to the invention on a panel of 50 people for 4 weeks. In the first test, the composition was used as make-up remover for the eyes and, in the second test, the composition was used as make-up remover for the face and for the eyes.

The results are as follows:

1) Test No. 1:

make-up-removing effectiveness of Example 3 as make-up remover for the eyes

Protocol:

Number of subjects: 50

Criteria for inclusion: Subjects of all skin types, including 50% using a waterproof make-up, 20% having sensitive eyes, 20% wearing contact lenses, 10% having normal eyes.

Duration of the treatment: 4 weeks

Area of application: eyes

Experimental scheme: removal of make-up once daily

Results

(the % values correspond to the percentages of people who have recorded the criterion)

| | % of people who |
|---|---|
| | have recorded |
| Criterion after 4 weeks of use | the criterion |
| Cosmetic criteria | |
| Gentle product for the eyes | 98.0% |
| Nongreasy product | 95.9% |
| Nontacky product | 98.0% |
| Leaves the eyes comfortable | 87.8% |
| Criteria of effectiveness | |
| Removes the waterproof make-up | 78.6% |
| Removes make-up completely from the eyes | 89.8% |
| Removes make-up gently | 98.0% |
| Removes make-up in a single movement | 49.0% |
| Removes make-up easily | 73.5% |
| Decongests tired eyelids | 59.2% |
| Does not dry out the skin of the outline | 91.8% |
| of the eye | |
| Leaves a feeling of freshness on the | 85.7% |
| eyes | |

The people who used the composition according to the invention assessed all the cosmetic criteria (87% to 98%), in particular the gentleness of the composition for the eyes and the nongreasy and nontacky aspect of the composition.

Furthermore, the people who used the composition according to the invention assessed the criteria of effectiveness (49% to 98%), in particular the removal of make-up gently, the absence of drying out the skin of the outline of the eye, the complete removal of make-up from the eyes and the feeling of freshness on the eyes. Approximately half of the subjects found that the composition removed the make-up in a single movement, which is not normal in the removal of make-up. Overall, the composition according to the invention obtained a very good assessment of its effectiveness on the sensitive area of the region of the eyes.

2) Test No. 2:

make-up-removing effectiveness of Example 3 as make-up remover for the face and eyes

Protocol:

Number of subjects: 42 people

Criteria for inclusion: Subjects having normal to combination skin, daily using make-up products for the face and eyes, including a minimum of 10 users of waterproof mascaras

Duration of the treatment: 1 week

Area of application: face and eyes

Experimental scheme: removal of make-up once daily, in the evening

Results:

| Overall satisfaction | Number of people |
|---|---|
| Very satisfactory | 11 out of 28 |
| Satisfactory | 8 out of 28 |
| Fairly satisfactory | 3 out of 28 |
| Rather unsatisfactory | 3 out of 28 |
| Unsatisfactory | 3 out of 28 |
| Very unsatisfactory | 0 |
| Overall mean grade out of 10 | 7.8 out of 10 |

Furthermore, the comfort was evaluated on the face and on the eyes:
for the comfort on the face, 25 people out of 28 found that the comfort on application was very good to fairly good and 21 people out of 28 found that the comfort after application was very good to fairly good,
for the comfort on the eyes, for the conventional mascara, 16 people out of 19 found that the comfort on application was very good to fairly good and 17 people out of 19 found that the comfort after application was very good to fairly good and, for the. waterproof mascara, 10 people out of 13 found that the comfort on application was very good to fairly good and 10 people out of 13 found that the comfort after application was very good to fairly good.

In addition, the make-up-removing effectiveness was also evaluated:
on the face, 23 people out of 28 found that the composition removed the make-up very rapidly to fairly rapidly,
on the eyes, for the conventional mascara, 17 people out of 19 found that the composition removed the make-up very rapidly to fairly rapidly and, for the waterproof mascara, 10 people out of. 13 found that the composition removed the make-up very rapidly to fairly rapidly.

These results show that a majority of the people who used the composition of the invention were satisfied with its effectiveness and with its comfort on use and the results confirm the excellent make-up-removing effectiveness on the face and the eyes of the composition according to the invention.

The above written description of the invention provides a manner and process of making and using it such that any person skilled in this art is enabled to make. and use the same, this enablement being provided in particular for the subject matter of the appended claims, which make up a part of the original description and including an aqueous make-up-removing cosmetic composition, wherein it comprises a derivative (product) of polyethylene glycol and of a mixture of glycerides of caprylic acid and of capric acid comprising 7 ethylene oxide groups in an amount of less than 10% by weight, with respect to the total weight of the composition.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. An aqueous composition, comprising water and a product of polyethylene glycol and a mixture of glycerides of caprylic acid and of capric acid comprising 7 ethylene oxide groups in an amount of less than or equal to 10% by weight, with respect to the total weight of the composition.

2. The composition according to claim 1, wherein it comprises at least 55% by weight of water, with respect to the total weight of the composition.

3. The composition according to claim 1, wherein the product of polyethylene glycol and a mixture of glycerides of caprylic acid and of capric acid comprising 7 ethylene oxide groups is present in an amount of 0.05 to 10% by weight, with respect to the total weight of the composition.

4. The composition according to claim 1, further comprising one or more surfactants other than a product of polyethylene glycol and a mixture of glycerides of caprylic acid and of capric acid comprising 7 ethylene oxide groups chosen from nonionic surfactants, anionic surfactants, amphoteric or zwitterionic surfactants and their mixtures.

5. The composition according to claim 4, wherein the surfactant or surfactants are chosen from oxyethylene/oxypropylene block polymers, polyoxyethylenated sorbitan esters, products of polyethylene glycol and a mixture of glycerides of caprylic acid and of capric acid having 6 or 8 ethylene oxide groups, and their mixtures.

6. The composition according to claim 1, further comprising a product of polyethylene glycol and a mixture of glycerides of caprylic acid and of capric acid having 6 ethylene oxide groups.

7. The composition according to claim 1, wherein the total amount of surfactant(s) present in the composition ranges from 0.01 to 12% by weight, with respect to the total weight of the composition.

8. The composition according to claim 1, in the form of a water for removing make-up from the skin and/or the region around the eyes.

9. The composition according to claim 1, which is compatible with the skin, mucous membranes, nails, region of the eyes and/or keratinous fibres.

10. The composition according to claim 1, wherein
the composition comprises at least 55% by weight of water, with respect to the total weight of the composition;
the product of polyethylene glycol and a mixture of glycerides of caprylic acid and of capric acid comprising 7 ethylene oxide groups is present in an amount of 0.05 to 10% by weight, with respect to the total weight of the composition;
the composition further comprises one or more surfactants other than a product of polyethylene glycol and a mixture of glycerides of caprylic acid and of capric acid comprising 7 ethylene oxide groups chosen from nonionic surfactants, anionic surfactants, amphoteric or zwitterionic surfactants and their mixtures; and
the composition is compatible with the skin, mucous membranes, nails, region of the eyes and/or keratinous fibres.

11. A method for removing make-up from the skin, comprising applying the composition according to claim 1 to skin which has makeup thereon.

12. A method for removing make-up from the region around the eyes, comprising applying the composition according to claim 1 to a region around the eyes which has makeup thereon.

13. A method for removing make-up from the skin, comprising applying the composition according to claim 9 to skin which has makeup thereon.

14. A method for removing make-up from the region around the eyes, comprising applying the composition according to claim 9 to a region around the eyes which has makeup thereon.

15. A method for removing make-up from the skin, comprising applying the composition according to claim 10 to skin which has makeup thereon.

16. A method for removing make-up from the region around the eyes, comprising applying the composition according to claim 10 to a region around the eyes which has makeup thereon.
